(19) United States
(12) Patent Application Publication
(10) Pub. No.: US 2023/0136890 A1
(43) Pub. Date: May 4, 2023

(54) INJECTION DEVICE HAVING A SUSPENDED PARENTERAL INTERFACE

(71) Applicant: West Pharmaceutical Services, Inc., Exton, PA (US)

(72) Inventors: Alex LYNESS, West Chester, PA (US); Geoffrey ROUIN, Morristown, NJ (US); Brian COSTELLO, Whitehouse Station, NJ (US)

(21) Appl. No.: 17/918,107

(22) PCT Filed: Mar. 23, 2021
(86) PCT No.: PCT/US2021/023685

§ 371 Date: Oct. 10, 2022

Related U.S. Application Data
(60) Provisional application No. 63/008,007, filed on Apr. 10, 2020.

(51) Int. Cl. A61M 5/142; A61M 5/158

(52) U.S. Cl. CPC A61M 5/14248; A61M 5/158; A61M 5/2033

(57) ABSTRACT

Systems and methods for drug delivery are disclosed in which an injection device comprises a parenteral interface, comprising a support and a needle assembly, suspended within a cavity in a housing of the device such that the parenteral interface is movably mounted with respect to the housing when the needle assembly is in the injection position. The parenteral interface can be configured to allow movement of the housing of the device with respect to the skin without displacing the needle assembly. The devices can be configured to passively or actively compensate for movement of the housing relative to the injection site to ensure correct delivery depth is maintained during time device is worn. For example, by mounting the support of the parenteral interface on a deformable mount or an articulated or rotatable component.

### CROSS-REFERENCE TO RELATED APPLICATION

This application claims priority to U.S. Provisional Application No. 63/008,007, filed on Apr. 10, 2020, the disclosure of which is incorporated herein by reference.

### TECHNICAL FIELD

The present disclosure relates generally to systems and methods for drug delivery comprising a ‘floating’ parenteral interface. In particular, the present disclosure relates to drug delivery devices and methods in which the parenteral interface is movably mounted with respect to a housing.

### BACKGROUND

Many medical conditions, both acute and chronic, can be managed or treated by parenteral delivery of medicament to a patient. Parenteral delivery methods include hypodermic injections to administer therapeutics agents into the body, for example by way of intradermal, subcutaneously, or intramuscular injections using a needle or cannula.

Wearable devices and autoinjectors can provide a convenient and safe method of delivering therapeutic agents. These are designed for convenience and comfort for the user and/or caregiver, and to ensure compliance with required dosage regimes. Many devices may be configured to reduce the visibility or manual processes associated with conventional delivery methods. They may also provide accurate dosage control to ensure consistent delivery and improve patient compliance.

One of the challenges associated with wearable devices and autoinjectors is maintaining appropriate contact and depth between the injection needle and the injection site throughout the duration of drug delivery. Dislodgement of the device during injection can cause pain and lead to incomplete delivery of medicament if the needle is dislodged from the injection site.

In addition to conventional hypodermic injection, therapeutic agents can be delivered through the skin via a hollow microneedle array. Microneedle arrays generally comprise an arrangement of multiple short, sharp structures which penetrate only the upper layer(s) of skin. Due to the reduced depth of insertion (and the reduced diameter of individual needles), microneedles tend to cause a reduced level of pain compared to conventional hypodermic needles. However, adoption of microneedle technology has been limited in some applications because the microneedle array must be applied at precise forces, and with an appropriate impact velocity, into the skin to achieve consistent microneedle depth and delivery of medicament without leakage. Detachment of the microneedle array from the injection site must also be avoided to prevent leakage of the drug through the microneedle array.

As a result, there is a need for a drug delivery device that ensures maintained contact between a needle or microneedle array and an injection site throughout the duration of therapeutic agent delivery. This is paramount given the amount of wearable drug delivery devices which will likely be worn for longer time periods by a user and in many different situations whether, active, dressing, resting, sleeping which may lead to the device casing being knocked whilst needle array is in the skin.

### SUMMARY

The present disclosure addresses some of the downsides associated with known injection devices, and in particular the manner in which the needle assembly of an injection device is attached and supported relative to the housing.

Aspects of the present disclosure provide various injection devices in which a parenteral interface, comprising a support and a needle assembly, is suspended within a cavity in a housing of the device such that the parenteral interface is movably mounted with respect to the housing when the needle assembly is in the injection position. The device can be a wearable device configured to be secured against the skin. The parenteral interface can be configured to allow movement of the housing of the device with respect to the skin without displacing the needle assembly.

The devices described herein can be configured to passively compensate for movement of the housing relative to the injection site (for example by mounting the support of the parenteral interface on a deformable mount or an articulated or rotatable component. Alternatively or additionally, devices described herein can comprise active compensation for movement of the housing, which can comprise a system configured to control the position of the needle assembly relative to the housing in response to sensed displacement of the housing or the parenteral interface from the skin. In some embodiments, active needle assembly positioning control can be configured to maintain the contact force between a needle assembly (e.g. a cannula or microneedle array) and an injection site.

In at least some embodiments, the parenteral interface can be configured to bias the needle assembly in a proximal direction (towards the injection site) such that, in the event of the housing being displaced in a distal direction away from the injection site, the parenteral interface is maintained in contact with the injection site. In these and other embodiments, the parenteral interface can be configured to allow lateral movement of the needle assembly relative to the housing in response to the housing being displaced laterally relative to the injection site. Alternatively or additionally, the parenteral interface can be configured to tilt within the cavity relative to the housing. Injection devices according to the present disclosure can therefore be generally considered to comprise: a housing configured to receive a medicament container; a parenteral interface comprising a needle assembly and a support arranged within a cavity in the housing. The support is mounted within the cavity such that the support can be displaced (e.g. tilted, rotated, or translated) relative to the housing whilst the parenteral interface is in an injection position.

By allowing movement of the needle assembly relative to the housing whilst the needle assembly is in the injection position, embodiments of the present disclosure may help to reduce pain and irritation for the user, reduce leakage of medicament from the injection site, and help to maintain the depth of needle insertion within a desired range throughout the duration of use of the device.

In a first aspect, there is provided an injection device comprising a housing configured to receive a medicament container and a parenteral interface comprising a support arranged within a cavity in the housing. A needle assembly is mounted to the support and configured to deliver a dose of medicament to an injection site. A flexible conduit is configured to deliver medicament from the medicament container to the needle assembly. The support is mounted on a plurality of springs coupled to a spring base provided in the housing. Each spring has a longitudinal axis and the longitudinal axes of the springs are non-coincident.

The spring base can be a portion of the housing, a component fixedly mounted within the housing or a needle insertion mechanism which is configured to advance the needle assembly relative to the housing to bring the needle assembly into contact with the injection site for delivery of an injection.

By mounting the parenteral interface on a plurality of springs, the parenteral interface can be configured to tilt, rotate, or translate relative to the housing to maintain (or assist in maintaining) the parenteral interface in contact with the injection site even during displacement of the device.

In a second aspect, there is provided an injection device comprising a housing configured to receive a medicament container and a parenteral interface comprising a support arranged within a cavity in the housing and a needle assembly mounted to the support and configured to deliver a dose of medicament to an injection site. A flexible conduit configured to deliver medicament from the medicament container to the needle assembly. The support is movably mounted within the cavity, and a plurality of motors is configured to move the support with respect to the housing to maintain the parenteral interface in contact with an injection site.

By mounting the parenteral interface on a plurality of motor driven actuators, the parenteral interface can be configured to tilt, rotate, or translate relative to the housing to maintain (or assist in maintaining) the parenteral interface in contact with the injection site even during displacement of the device.

In a third aspect, there is provided an injection device comprising a housing configured to receive a medicament container and a parenteral interface. The parenteral interface comprises a support and a needle assembly mounted to the support and configured to deliver a dose of medicament to an injection site, the support being arranged within a cavity within the housing. A flexible conduit is configured to deliver medicament from the medicament container to the needle assembly. The support is rotationally mounted with respect to the housing to allow rotation of the support within the cavity.

The rotational mount for the support can be configured to allow at least one of rotation about an axis substantially perpendicular to the surface of the injection site (thereby allowing the support to twist relative to the housing. The rotational mount for the support can also be configured to allow rotation about multiple axes. For example, the rotational mount may allow for twisting relative to the housing, and tilting of the support within the cavity.

By mounting the parenteral interface on a rotational mount, the parenteral interface can be configured to tilt, rotate, or translate relative to the housing to maintain (or assist in maintaining) the parenteral interface in contact with the injection site even during displacement of the device.

In a fourth aspect, there is provided an injection device comprising a housing configured to receive a medicament container and a parenteral interface. The parenteral interface comprises a support and a needle assembly mounted to the support and configured to deliver a dose of medicament to an injection site, the support being arranged in a cavity within the housing. A flexible conduit is configured to deliver medicament from the medicament container to the needle assembly. The support is mounted within the cavity on a resiliently deformable mount coupled to the housing. The support comprises an outer sidewall, the cavity comprises an inner sidewall, and the outer sidewall of the support is separated from the inner sidewall of the cavity by a circumferential space extending around the outer sidewall of the support.

In any of the aspects described above, the needle assembly can comprise a hollow injection needle. Alternatively (or additionally), the needle assembly can comprise a microneedle array.

The housing can include a skin contacting surface, the skin contacting surface comprising an adhesive portion configured to attach the device to the skin of a user. Additionally or alternatively, a further adhesive portion can be provided on a skin facing surface of the support. In at least some embodiments, the injection device is a wearable injection device and comprises a securing means for securing the device on a user's body.

The device can further include a mount coupled to the support. The mount may take the form of the spring base, the deformably mount or the rotational mount of the aspects described above. The mount can be movably mounted within the housing for travel in a proximal direction to advance the support and thus the needle assembly from a retracted position to an extended position from the device housing towards the skin.

The device can further comprise an insertion mechanism configured to move the support and/or the mount (e.g. the spring base, the deformable mount or the rotational mount) between a first position relative to the housing in which the needle assembly does not extend from the housing, and a second position relative to the housing, in which the needle assembly extends from the housing for insertion into an injection site. The support and/or the mount can be mounted within the housing for travel in a proximal direction to advance the needle assembly from a retracted position to an extended position from the device housing towards the skin.

Optionally, the injection device further comprises a releasable locking mechanism configured to: (i) maintain the mount and/or the support in a first position relative to the housing, in which the needle assembly does not extend from the housing, when the locking mechanism is in an active state; and to allow movement of the support and/or the mount relative to the housing when the locking mechanism is in an inactive state.

In one configuration, the releasable locking mechanism can be configured to: (i) maintain the support in a first position relative to mount in which the plurality of springs (or the deformable material) are compressed, and in which the needle assembly does not extend from the housing, when the locking mechanism is in an active state; and (ii) allow movement of the support relative to the mount under the influence of the plurality of springs (or the deformable material) to a second position, in which the needle assembly extends from the housing, when the locking mechanism is in an inactive state.

In some embodiments, the device further comprises a deployment mechanism coupled to the support, wherein the deployment mechanism is configured to move the support between a first position in which the needle assembly does not extend from the housing and a second position in which the needle assembly extends from the housing. Optionally, the deployment mechanism can be removably coupled to the housing.

In any of the aspects and embodiments describe above, the device can comprise one or more of the following mounts: a plurality of springs, a resiliently deformably mount, a rotationally or pivotally mounted support, a motor actuated support or any combination of the above. For example, a pivotally mounted support may be mounted on a plurality of springs to allow the support to twist relative to the housing (with minimal resistance) whilst the plurality of springs bias the parenteral interface in the proximal direction to maintain contact between the needle assembly and the injection site. In another example, a motor actuated support can be combined with a deformable mount to provide additional comfort to the user and to help maintain even pressure at the injection site.

Accordingly, the plurality of springs can include at least one helical spring. The plurality of springs can be fixed to a spring base. The spring base can be movably mounted in the housing. The spring base can be rotatably mounted in the housing. Alternatively or additionally, the spring base can be pivotably mounted the housing. In yet further embodiments, in combination with or as an alternatively to rotationally and pivotably mounting components, the spring base can be movably mounted within the housing for travel in a proximal direction to advance the needle assembly from a retracted position to an extend position from the device housing towards the skin. In any embodiment, the plurality of springs can be configured to bias the support into a position in which the needle extends beyond a skin contacting surface of the housing.

The resiliently deformable mount can comprise a foam layer. The foam layer can be fixed to a base. The base can be movable relative to the housing for travel in a proximal direction to advance the needle assembly from a retracted position to an extend position from the device housing towards the skin. Alternatively, the foam layer can be fixedly mounted within the housing. The spring base can be rotatably mounted in the housing. Alternatively or additionally, the spring base can be pivotably mounted the housing.

Any of the embodiments described above can be provided with a motor (or a plurality of motors) and at least one corresponding actuator configured to move the support relative to the housing to maintain a contact between the parenteral interface and the skin at the injection site. The motor(s) can be a servo motor. Each of the motors may be in communication with at least one sensor configured to sense detachment of the parenteral interface from an injection site. Optionally, the at least one sensor is a microneedle electrode sensor. The sensor(s) can be provided on a skin contacting surface of the housing. Additionally or alternatively, at least one sensor can be provided on a skin facing surface of the support. The motors can be mounted to a motor base, which is movably mounted within the housing. For example, the motor base may be rotatably mounted in the housing and/or pivotably mounted within the housing. In some configurations, the base may be movably mounted within the housing for travel in a distal direction to advance the needle assembly from a retracted position to an extended position towards the skin. In any of the configurations described above, the plurality of motors can be configured to maintain the support in a position in which the needle extends beyond a skin contacting surface of the housing.

In any of the aspects and embodiments describe above, the support can be rotationally mounted with respect to the housing. The support may be rotationally mounted to a fixed component, such as the housing or a mount, or the support may be fixedly mounted to a mount, which is rotationally mounted with respect to the housing.

Any of the embodiments described above may further comprise a swivel joint configured to allow rotation of the support within the housing about an axis extending in a proximal direction from the device housing towards the skin. The support can be configured to pivot about a pivot point. In some embodiments, the support may be mounted with respect to the housing with a ball and socket joint. The support can be rotationally mounted to a support base, which is movably mounted within the housing for travel in a distal direction to advance the needle assembly from a retracted position to an extend position. The rotatable support described above can further comprise a plurality of springs (or other biasing means) configured to bias the support into a position in which the needle extends beyond a skin contacting surface of the housing.

The support may be disposed within the cavity of the housing to allow at least one of lateral, proximal, rotational or pivotal movement of the support relative to the housing. In some configurations, the cavity can be configured to allow lateral displacement of the support relative to the housing. In such embodiments, the support can comprise an outer sidewall, the cavity can comprise an inner sidewall, and the outer sidewall of the support may be separated from the inner sidewall of the cavity by a circumferential space extending around the outer sidewall of the support. For example, the foam layer may be separated from the inner side wall of the cavity by a circumferential space.

The present disclosure also provides associated methods of supporting a needle of an injection device in an injection position, and methods of manufacturing an injection device according to any of the aspects or embodiments described above.

Accordingly, in a fifth aspect, there is provided a method of manufacturing an injection device, comprising the steps of: providing a housing configured to receive a medicament container, the housing further comprising a cavity having an opening on a skin contacting surface of the housing; providing, within the cavity of the housing, a parenteral interface comprising a support and a needle assembly mounted to the support and configured to deliver a dose of medicament to an injection site, the parenteral support being arranged in a cavity within the housing; providing a flexible conduit configured to deliver medicament from the medicament container to the needle assembly; and mounting the support within the cavity on a plurality of motors configured to maintain contact between the parenteral interface with an injection site.

In a sixth aspect, there is provided a method of manufacturing an injection device, comprising the steps of: providing a housing configured to receive a medicament container, the housing further comprising a cavity having an opening on a skin contacting surface of the housing; providing, within the cavity of the housing, a parenteral interface comprising a support and a needle assembly mounted to the support and configured to deliver a dose of medicament to an injection site, the parenteral support being arranged in a cavity within the housing; providing a flexible conduit configured to deliver medicament from the medicament container to the needle assembly; and mounting the support on a plurality of springs coupled to a spring base provided in the housing, each spring having a longitudinal axis, and wherein the longitudinal axes are non-coincident.

In a seventh aspect, there is provided a method of manufacturing an injection device, the method comprising the steps of: providing a housing configured to receive a medicament container, the housing further comprising a cavity having an opening on a skin contacting surface of the housing; providing, within the cavity of the housing, a parenteral interface comprising a support and a needle assembly mounted to the support and configured to deliver a dose of medicament to an injection site, the parenteral support being arranged in a cavity within the housing; providing a flexible conduit configured to deliver medicament from the medicament container to the needle assembly; and mounting the support within the cavity, wherein the support is rotationally mounted with respect to the housing to allow rotation of the support about at least one axis relative to the housing.

In an eighth aspect, there is provided a method of manufacturing injection device, comprising the steps of: providing a housing configured to receive a medicament container, the housing further comprising a cavity having an opening on a skin contacting surface of the housing; providing, within the cavity of the housing, a parenteral interface comprising a support and a needle assembly mounted to the support and configured to deliver a dose of medicament to an injection site, the parenteral support being arranged in a cavity within the housing; providing a flexible conduit configured to deliver medicament from the medicament container to the needle assembly; and mounting the support within the cavity on a resiliently deformable mount coupled to the housing, wherein the support comprises an outer sidewall, the cavity comprises an inner sidewall, and the outer sidewall of the support is separated from the inner sidewall of the cavity by a circumferential space extending around the outer sidewall of the support.

The methods of the fifth, sixth, seventh and eighth aspects may further comprise the steps of providing any of the features described above with reference to the first to fourth aspects.

In a ninth aspect, there is provided a method of supporting a needle of an injection device in preparation for injection of a medicament, the method comprising: placing, against an injection site, a device comprising: a housing having a cavity, a parenteral interface arranged within the cavity, wherein the parenteral interface comprises a support and a needle assembly mounted to the support and configured to deliver a dose of medicament to an injection site, a flexible conduit configured to deliver medicament from the medicament container to the needle assembly, wherein the support is movably mounted within the cavity on a plurality of motors configured to maintain contact between the parenteral interface with an injection site, fixing the injection device to the injection site using an adhesive provided on the housing; maintaining contact between the parenteral interface and the injection site by actuation of at least one motor. Optionally, the plurality of motors is in communication with at least one sensor configured to detect detachment of the parenteral interface from an injection site, and wherein the motors are configured to actuate in response to sensed detachment of the parenteral interface from the injection site.

In a tenth aspect, there is provided a method of supporting a needle of an injection device in preparation for injection of a medicament, the method comprising: placing, against an injection site, a device comprising: a housing having a cavity, a parenteral interface arranged within the cavity, wherein the parenteral interface comprises a support and a needle assembly mounted to the support and configured to deliver a dose of medicament to an injection site, wherein the support is mounted on a plurality of springs extending from a spring base within the housing, the plurality of springs having non-coincidental longitudinal axes; fixing the injection device to the injection site using an adhesive provided on the housing; and compressing the springs between the support and the spring base.

In an eleventh aspect, there is provided a method of supporting a needle of an injection device in preparation for injection of a medicament, the method comprising: placing, against an injection site, a device comprising: a housing having a cavity, a parenteral interface arranged within the cavity, wherein the parenteral interface comprises a support and a needle assembly mounted to the support and configured to deliver a dose of medicament to an injection site, wherein a flexible conduit is provided in fluid communication with the medicament container and the needle assembly, and wherein the support is rotationally mounted with respect the housing to allow rotation of the support within the cavity, fixing the injection device to the injection site using an adhesive provided on the housing, and bringing the parenteral interface into contact with an injection site.

In a twelfth aspect, there is provided a method of supporting a needle of an injection device in preparation for injection of a medicament, the method comprising: placing, against an injection site, a device comprising: a housing having a cavity, a parenteral interface arranged within the cavity, wherein the parenteral interface comprises a support and a needle assembly mounted to the support and configured to deliver a dose of medicament to an injection site, wherein the support is mounted within the cavity on a resiliently deformable mount coupled to the housing and wherein: the support comprises an outer sidewall; the cavity comprises an inner sidewall, and the outer sidewall of the support is separated from the inner sidewall of the cavity by a circumferential space extending around the outer sidewall of the support; fixing the injection device to the injection site using an adhesive provided on the housing; and compressing the resiliently deformable material between the support and the housing.

Further advantages and additional embodiments will be apparent from the following detailed description and the accompanying drawings.

### BRIEF DESCRIPTION OF THE DRAWINGS

The invention will be described in more detail with reference to a number of non-limiting, exemplary embodiments shown in the following drawings, in which:

FIG. 1A shows a schematic side view of a wearable injection device placed against an injection site;

FIG. 1B shows a side view of an autoinjector placed against an injection site;

FIG. 10 shows a schematic top view of a wearable injection device placed against an injection site;

FIG. 2A shows a cross-sectional side view of an injection device according to a first embodiment;

FIG. 2B shows the injection device of FIG. 2A during an impact;

FIG. 3A shows a cross-sectional side view of an injection device according to a second embodiment;

FIG. 3B shows the injection device of FIG. 3A during an impact;

FIG. 4A shows a cross-sectional side view of an injection device according to a third embodiment;

FIG. 4B shows the injection device of FIG. 4A during an impact;

FIG. 5A shows a cross-sectional side view of an injection device according to a fourth embodiment;

FIG. 5B shows the injection device of FIG. 5A during an impact;

FIG. 6A shows a cross-sectional side view of an injection device according to a fifth embodiment;

FIG. 6B shows the injection device of FIG. 6A during an impact;

FIG. 7A shows a cross-sectional side view of an injection device according to a sixth embodiment;

FIG. 7B shows the injection device of FIG. 7A during an impact;

FIG. 8 shows a cross-sectional side view of an injection device according to a seventh embodiment;

FIG. 9 shows a cross-sectional side view of an injection device according to an eighth embodiment;

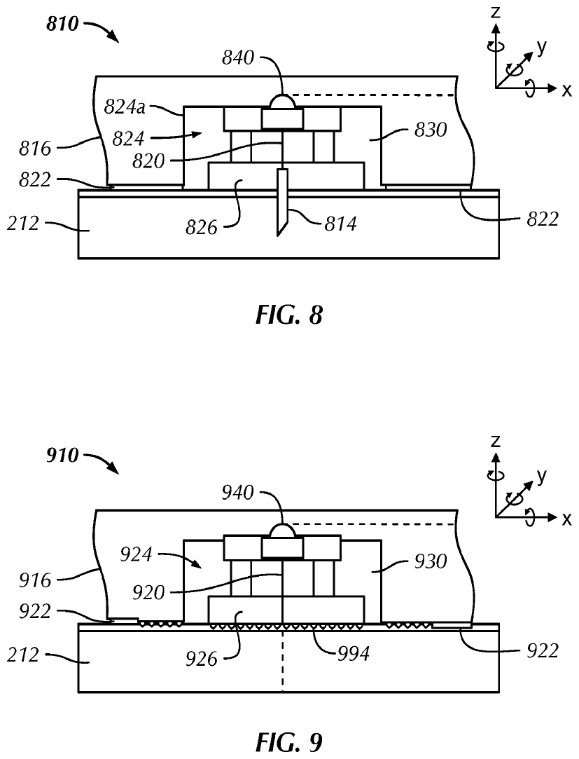

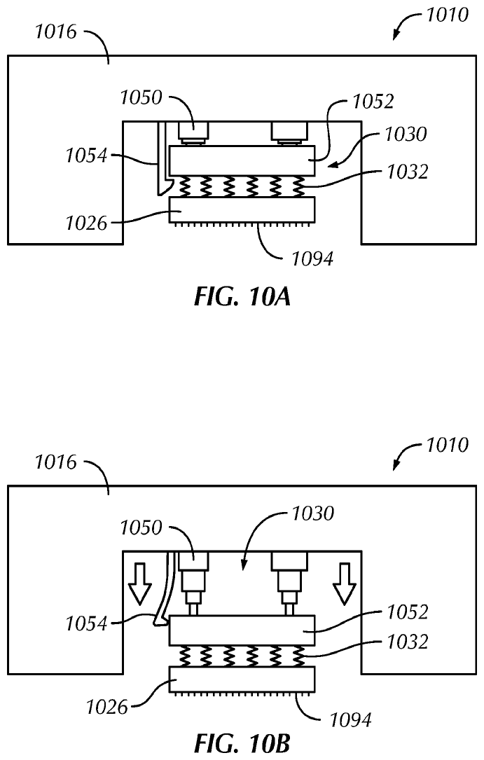

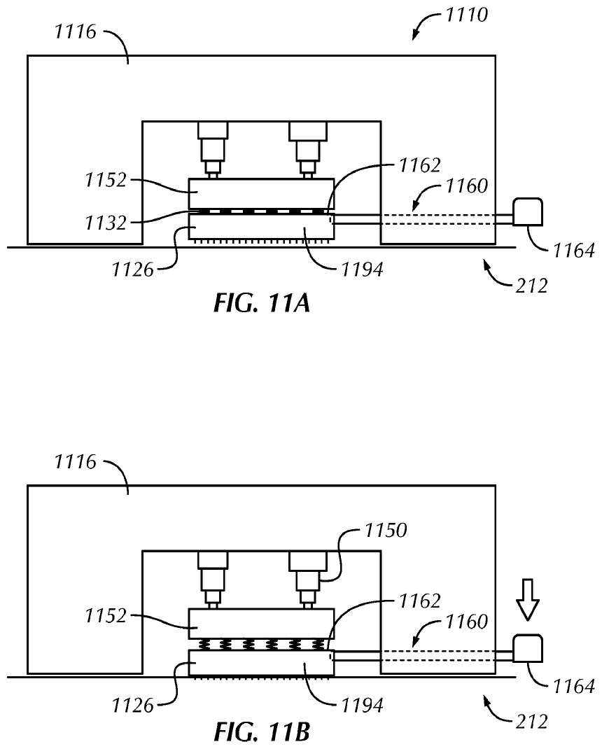

FIG. 10A shows a cross-sectional side view of an injection device according to a ninth embodiment with the needle assembly in a retracted position;

FIG. 10B shows a cross-sectional side view of the injection device of FIG. 10A with the needle assembly in an extended position;

FIG. 11A shows a cross-sectional side view of an injection device according to a tenth embodiment with the needle assembly in a retracted position;

FIG. 11B shows a cross-sectional side view of the injection device of FIG. 11A with the needle assembly in an extended position.

Like reference numerals are used for like components throughout the drawings.

### DETAILED DESCRIPTION

The present disclosure is directed generally to systems and methods for supporting a parenteral interface in an injection position. In particular, systems and methods described herein are configured to maintain the position and depth of a needle assembly on the surface of the skin in the event of displacement of the housing of the injection device.

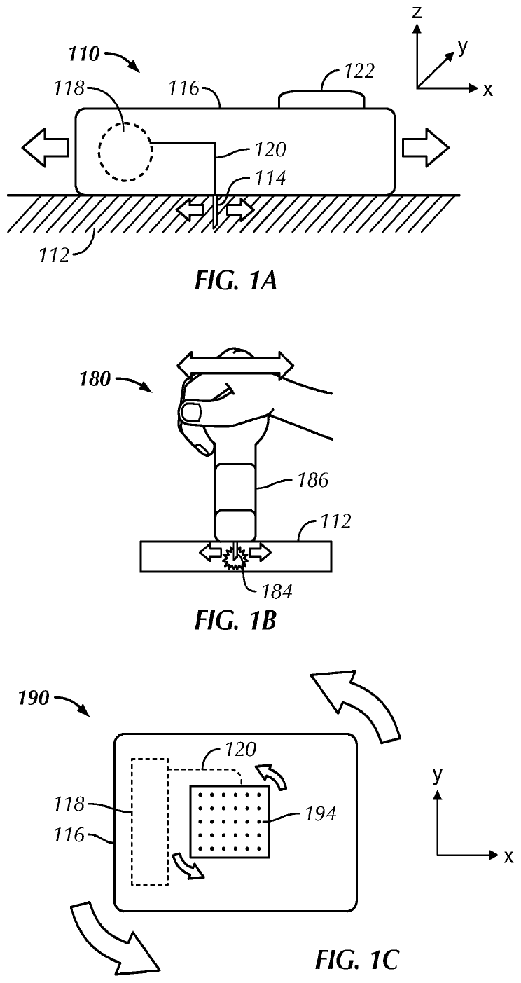

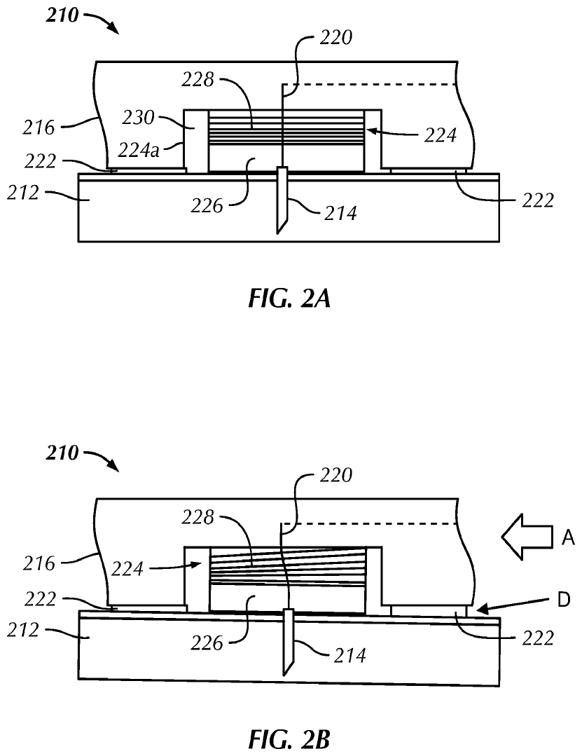

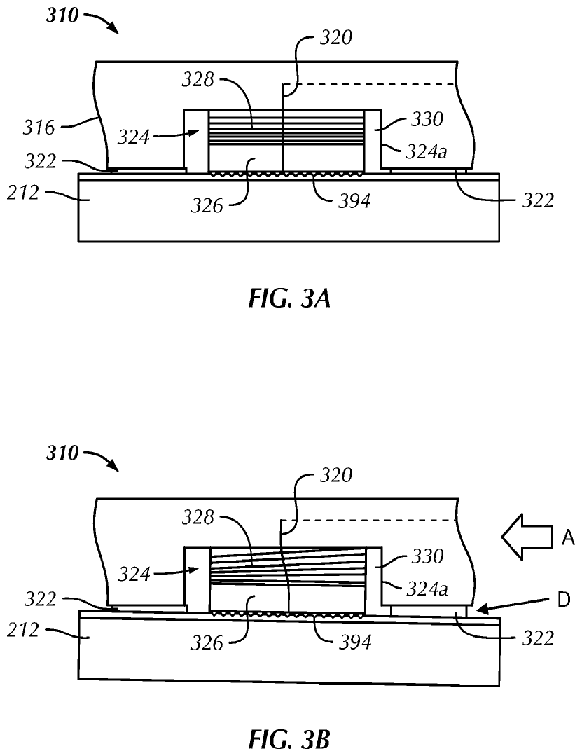

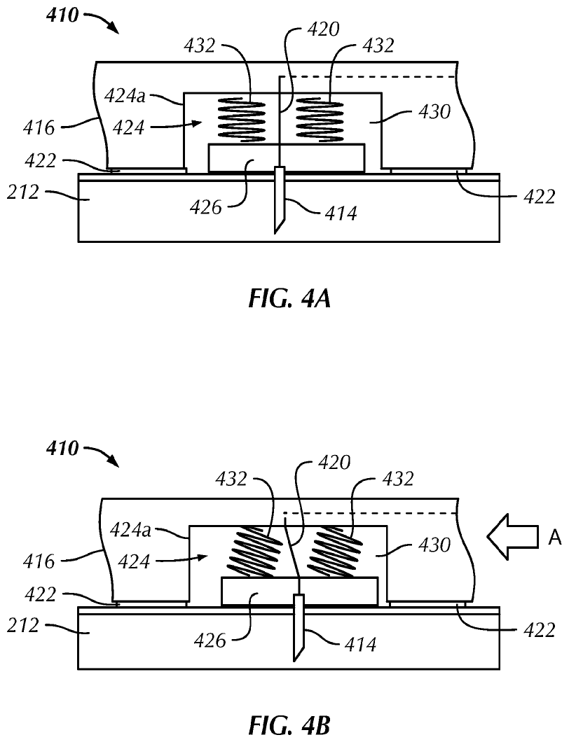

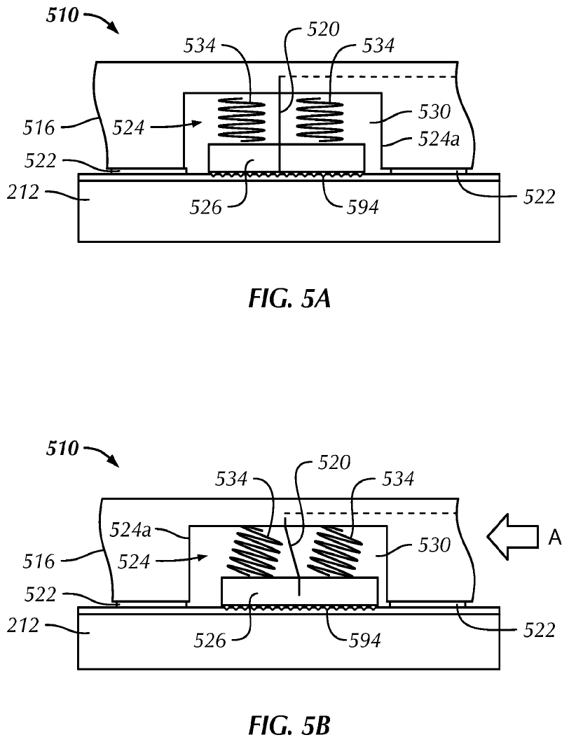

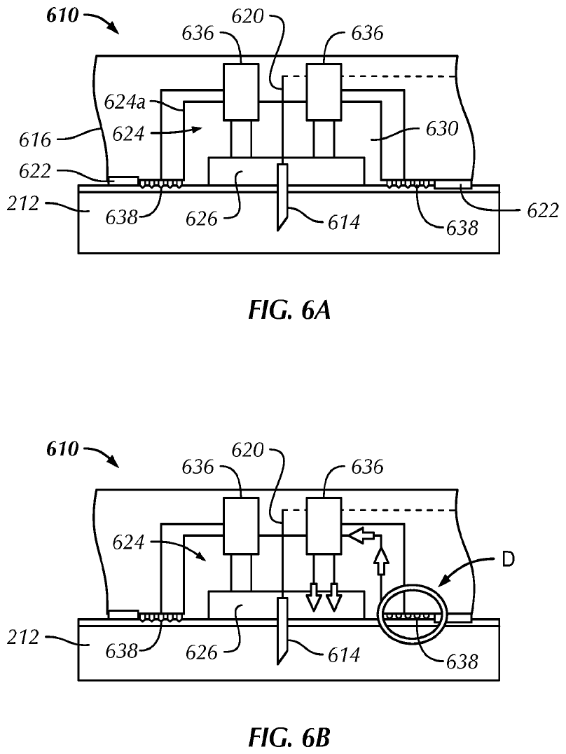

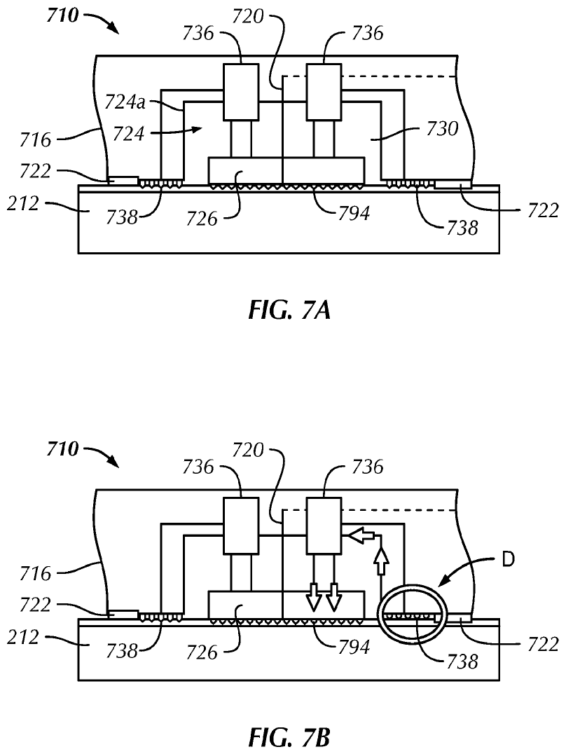

FIG. 1A shows a schematic side view of a wearable injection device placed against an injection site. The injection device 110 is placed against the skin 112 and secured in place with an adhesive or other attachment means. A needle 114 extends from a housing 116 of the injection device and penetrates the skin for delivery of a therapeutic agent to the patient. Typically (although not always), the housing 116 of the device 110 also includes a container 118 for storing medicament and a conduit 120 providing fluid communication between the container 118 and the needle 114. A drive system (not shown) is also typically housed in the housing 116 and is configured to drive expulsion of medicament from the container 118 through the conduit 120 for delivery through the needle 114. The drive system can comprise a mechanical drive (e.g. a spring or clockwork mechanism) and/or an electric drive (e.g. a motor) configured to deliver the medicament. An actuator, for example button 122, may be provided on the housing 116 so that the user can trigger the start of injection. The skilled person will appreciate however that a mechanical actuator is not necessary and the device can be controlled and actuated electronically, for example using a remote device in wireless communication with the drive system.

In the device shown in FIG. 1A, the needle 114 is fixedly mounted with respect to the housing 116, generally for at least the duration of the injection. This means that if the housing 116 is knocked, causing lateral displacement of the housing 116 with respect to the skin 112, the needle 114 is also laterally displaced, which can cause pain and irritation for the user. Lateral displacement (in the x-y plane) of the housing 116 can compromise the connection between the needle 114 and the conduit 120 or dislodgment of the needle 114 from the injection site, which may lead to leakage of medicament from the device and poor dosage control.

Although not illustrated in FIG. 1A, it will also be appreciated that axial displacement of the housing 116 (in the z direction) can also alter the depth of needle insertion. This in turn may adversely impact the device's ability to correctly deliver the drug to the patient.

FIG. 1B illustrates how similar problems can occur with traditional autoinjectors. FIG. 1B shows an autoinjector 180 placed against the skin 112 of an injection site during injection. As shown in FIG. 1B, the autoinjector 180 comprises a housing 186 and a needle 184. Because of the fixed position of the needle 184 with respect to the housing 186 during injection, movement of the housing 186 relative to the injection side during injection results in movement of the needle 184, which can cause pain or lead to sub-optimal delivery of medicament. However, as instances of drug delivery with an auto-injector are typically 10-120 seconds and controlled by a patient, they can self-align the needle to make the delivery more comfortable and or withdraw the needle is heavily knocked.

FIG. 10 illustrates another wearable injection device 190 similar to the device 110 of FIG. 1A. However, whereas the device 110 of FIG. 1A comprises a traditional cannula to deliver medicament to the injection site, the device 190 of FIG. 10 comprises a hollow microneedle array (MNA) 194. The microneedle array 194 is arranged within a cavity in the underside of the housing 116 (similar to the cavity shown in FIG. 1A) and is provided in fluid communication via conduit 120 with container 118.

In the device of FIG. 10, the microneedle array 194 is fixedly mounted with respect to the housing 116 of the device (during injection) and so any impact that causes rotation (e.g. in the x-y plane) of the housing 116 relative to the skin of the injection site can result in twisting of the microneedle array 194 relative to the skin. This rotational displacement can cause pain and irritation for the user, but may also lead to detachment of the microneedle array 194 from the injection site of the correct depth, causing leakage of medicament and poor dosage control.

In each of the configurations described above, displacement of the housing of the injection device relative to the injection site also results in displacement of the needle assembly relative to the injection site, risking pain and discomfort for the user, irritation or injury of the injection site over time, and sub-optimal drug delivery. Turning now to FIGS. 2A-9, at least some of the downsides described above may be addressed or reduced by embodiments of the present disclosure. In general, each of the embodiments described below comprises an injection device having a parenteral interface, which comprises a needle assembly configured to deliver medicament to an injection site, and a support configured to support the needle assembly in a position in which it places the needle assembly in an injection-ready position. The support is movably mounted with respect to the housing to allow the parenteral interface to move relative to the housing during injection to isolate (or insulate) the parenteral interface from impacts to the housing. The support may be movably mounted or a deformably or pliable material which couples the support to the housing, or its position may be actively controlled using actuators configured to move the support relative to the housing in response to sensed or detected movement of the housing relative to the injection site. In some embodiments, the device can be configured to actively control the position of the support relative to the housing in response to sensed dislodgment of the housing or the needle assembly (or reduced contact pressure). In other embodiments, the parenteral interface can comprise a resiliently deformable support component configured to deform as the housing is displaced related to the injection site, to isolate the parenteral interface from displacement of the housing and allow the parenteral interface to maintain its position in the injection site. Allowing movement of the parenteral interface with respect to the housing of the injection device can allow the needle penetration depth to be maintained throughout the course of an injection, even if the housing of the device were to be displaced relative to the skin (e.g. by knocking a wearable device or by an unsteady grip on a hand-held device). Allowing the parenteral interface to be independent on a sub-assembly or support that is suspended or floating relative to the housing of a delivery system can mitigate discomfort caused by dislodging of a device from its original position on the surface of the body.

FIGS. 2A and 2B show a first embodiment of an injection device according to the disclosure. FIG. 2A shows the injection device in its undisturbed seating position against the injection site. As shown in FIG. 2A, the injection device 210 comprises a housing 216 which is configured to be placed against the skin 212 of an injection site. The device also comprises a needle assembly, which here takes the form of a hollow injection needle 214 configured to deliver medicament from a container (not shown) through conduit 220 to the injection site. An adhesive layer 222 is provided to secure the injection device 210 to the skin 212. The adhesive layer 222 is provided on an underside surface (that is a skin facing surface) of the housing 216. The housing 216 comprises a cavity 224 in which the needle assembly is arranged. The needle assembly forms part of a parenteral interface which is configured to deliver medicament from the medicament container (not shown) to the needle 214. The parenteral interface comprises the needle 214 and a support 226 on which the needle 214 is mounted. The support 226 is mounted with respect to the housing 216 on a resiliently deformable mount 228. In the embodiment shown in FIG. 2A, the resiliently deformable mount 228 comprises a pliable foam layer.

The conduit 220 which connects the container (not shown) to the needle 214 is flexible along at least a portion of its length. The flexibility of the conduit is provided to allow the needle 214 to move relative to the housing 216, as will now be described in more detail with reference to FIG. 2B.

FIG. 2B shows the device 210 of FIG. 2A during an impact that laterally displaces the housing 216 relative to the injection site. The displacement of the housing 216 relative to the skin 212 is indicated with arrow A. As the housing 216 is displaced, the adhesive layer 222 attaching the device 210 to the skin 212 stretches. Detachment of the housing 216 from the skin 212 may also occur if the housing 216 is lifted away from the skin 212 (for example, at the point located with arrow D).

As shown in FIG. 2B, the pliable foam layer 228 is configured to deform to allow movement of the housing 216 relative to the injection site without displacing the needle 214. The flexible conduit 220 also allow movement of the needle 214 and the support 226 relative to the housing, allowing the conduit to accommodate movement of the needle 214 relative to the housing without interrupting the supply of medicament through conduit 220.

Because the parenteral interface (comprising the support 226 and the needle 214) is not rigidly connected to the housing 216, displacement of the housing 216 relative to the injection site does not displace (or displaces to a lesser extent) the needle 214. Instead, the pliable foam layer 228 and the flexible conduit 220 allow the parenteral interface to remain attached to the injection site whilst the housing 216 is displaced (laterally or otherwise).

In the embodiment shown in FIGS. 2A and 2B, the housing 216 is secured to the skin 212 with an adhesive layer 222 provided on an underside of the housing 216. The parenteral interface is maintained in contact with the skin 212 during injection due to its position with respect to the housing 216. In at least some embodiments, the support 226 may also comprise an adhesive layer (not shown in FIGS. 2A and 2B) configured to maintain contact between the parenteral interface and the injection site. Such an adhesive layer may be provided on the support 226 of the parenteral interface, on a skin facing surface of the support 226. Although an adhesive layer provided on the parenteral interface may be advantageous in some implementations, this feature is optional. The insertion of the needle 214 into the skin acts to prevent lateral displacement of the needle 214 relative to the injection site.

Although not indicated with arrows in FIGS. 2A and 2B, the pliable foam layer 228 may also act to prevent variation in the depth of needle penetration within the injection site. This is possible due to the pliable nature of the foam layer, which can be configured for compression and expansion in the z direction (the z direction being the direction along the axis of the needle 214. The compression or expansion of the pliable foam layer can also be configured to assist biasing of the needle 214 into the injection position. For example, the pliable foam layer 228 can be configured to bias the support 226 into a position in which a skin contacting surface of the support 226 projects beyond the underside of the housing 216. Placement of the injection device 210 against the skin 212 can act to compress the pliable foam layer 228. By configuring the injection device 210 such that the pliable foam layer is slightly compressed with the device in the position shown in FIG. 2A, the pliable foam layer can be configured to expand in the event that the housing 216 is pulled away from the injection site, thereby maintaining the depth of insertion of the needle 214, or at least providing a buffer against withdrawal of the needle 214 from the injection site.

To allow lateral displacement of the parenteral interface (comprising the support 226 and the needle 214) relative to the housing 216 (as shown in FIG. 2B), a circumferential space 230 is provided between the support 226 and a side wall 224a of the cavity 224. It will be appreciated that a circumferential space 230 allows a rigid support 226 to move laterally within a cavity 224 having rigid walls. However, the circumferential space 230 may be omitted in some embodiments, for example where the support has a diameter that is less than the pliable foam layer, or where the support is also formed of a pliable material.

FIGS. 3A and 3B show a second embodiment of an injection device according to the disclosure. The injection device 310 shown in FIGS. 3A and 3B is similar to the injection device 210 shown in FIGS. 2A and 2B. Like FIG. 2A, FIG. 3A shows the device 310 in a rest position (a non-displaced position). FIG. 3B shows the device 310 during an impact that displaces the device 310 relative to the skin 212 of the injection site.

The device 310 comprises a housing 316 configured to be secured in place against the skin using an adhesive layer 322 or adhesive patches. The housing 316 comprises a cavity 324, which houses a parenteral interface comprising a support 326 mounted on a pliable foam layer 228 (or another suitable resiliently deformable mount). The cavity comprises sidewalls 324a, which are separated from the support 326 by an optional circumferential space 330. Where the device 310 differs from the device 210 of FIGS. 2A and 2B is in the form that the needle assembly takes. In the embodiment described with reference to FIGS. 2A and 2B, the parenteral interface comprises a single needle 214 mounted on the support 226. However, in the embodiment shown in FIGS. 3A and 3B, the needle assembly takes the form of a microneedle array 294, which comprises a plurality of microneedles configured to deliver medicament to the injection site. The flexible conduit 320 is therefore configured to supply the microneedle array 394 in place of being connected to a single hollow injection needle, as shown in FIGS. 2A and 2B.

Like the embodiment shown in FIGS. 2A and 2B, when the housing 316 is displaced relative to the injection site, the pliable foam layer deforms to allow movement of the housing 316 relative to the injection site whilst allowing the microneedle array 394 to remain in situ.

It will be appreciated that by mounting the support of the embodiments described above on a resiliently deformable mount, such as a pliable foam layer, the support can be movably mounted relative to the housing to isolate the parenteral interface from impacts to the housing. Although the embodiments described above are depicted with a pliable foam layer, other deformable materials may also be used. For example, gel-based, or rubber-based layers may also be used. Moreover, it is also envisaged that the deformable mount can comprise a layer of material, or a plurality of discrete portions of material. For example a plurality of deformable portions may form the deformable mount, placed adjacent (and optionally abutting) each other, or with spaced disposed in between discrete portions of deformable material. Pliable foam may be a particularly well suited to some implementations in which ease of assembly is of paramount assembly. For example, the support may be glued in place on a pliable material layer, which may itself be glued to a component within the housing.

It will also be appreciated that the deformable mount described above does not need to be statically mounted respect to the housing. For example, the features described above may be implemented in injection devices comprising a movable needle hub which is configured to advance the needle from a retracted, pre-injection position (in which the needle does not extend from the housing) to an advanced, injection position (in which the needle extends from the housing to penetrate an injection site). In such embodiments, the resiliently deformable mount may be provided between the support and the needle hub component.

Turning now to FIGS. 4A and 4B, a third embodiment according to the disclosure will be described. The embodiment shown in FIGS. 4A and 4B is largely similar to the embodiment described with reference to FIGS. 2A and 2B. Like FIG. 2A, FIG. 4A shows the device 410 in a rest position (a non-displaced position). FIG. 4B shows the device 410 during an impact that displaces the device 410 relative to the skin 212 of the injection site.

As shown in FIG. 4A, the device 410 comprises a housing 416 configured to be secured against the skin 212 on an injection site with an adhesive 422. The housing 416 comprises a cavity 424 configured to house a parenteral interface that comprises a support 426 and a needle assembly, which here takes the form of a hypodermic needle 414. The needle 414 is provided is configured to be placed in fluid communication with a medicament container (not shown) via a flexible conduit 420. The cavity 424 comprises a side wall 424a and a circumferential gap 430 that extends between an outer surface of the support 426 and the side wall 424a of the cavity. The circumferential gap 430 allows lateral movement of the support 426 within the cavity 424 to allow displacement of the needle 414 relative to the housing 416 of the device 410.

Whereas the support 226 of device 210 is mounted on a pliable foam layer, in the embodiment shown in FIG. 4A, the support 426 is mounted on a plurality of springs 432, which are configured to deform to allow the support 426 to move relative to the housing 416, as shown in FIG. 4B. The springs 432 are arranged such that they are non-coaxial with respect to each other. The springs 432 are further advantageously arranged symmetrically with respect to the needle 414 to ensure even application of force in the z direction of the needle 414 into the injection site. The non-coaxial arrangement can allow the support 426 to be mounted stably on a plurality of smaller diameter springs, which allow lateral movement of the support 426 relative to the housing 416.

In some embodiments, the support 426 may be mounted on two springs arranged symmetrically with respect to the needle 414, such as on opposing sides. In other embodiments, one, three, four or more springs can be provided, arranged non-coaxially with respect to each other to movably support the support 426 relative to the housing.

FIGS. 5A and 5B show a fourth embodiment of an injection device according to the disclosure. The injection device 510 shown in FIGS. 5A and 5B is similar to the injection device 410 shown in FIGS. 4A and 4B. Like FIG. 4A, FIG. 5A shows the device 510 in a rest position (a non-displaced position). FIG. 5B shows the device 510 during an impact that displaces the device 510 relative to the skin 212 of the injection site.

The device 510 comprises a housing 516 configured to be secured in place against the skin using an adhesive layer 522 or adhesive patches. The housing 516 comprises a cavity 524, which houses a parenteral interface comprising a support 526 mounted on a plurality of springs 534. The cavity comprises sidewalls 324a, which are separated from the support 526 by an optional circumferential space 530. Where the device 510 differs from the device 510 of FIGS. 4A and 4B is in the form that needle assembly takes. In the embodiment described with reference to FIGS. 4A and 4B, the parenteral interface comprises a needle 414 mounted on the support 426. However, in the embodiment shown in FIGS. 5A and 5B, the needle assembly takes the form of a microneedle array 594, which comprises a plurality of microneedles configured to deliver medicament to the injection site. The flexible conduit 520 is therefore configured to supply the microneedle array 594 in place of being connected to a single hollow injection needle, as shown in FIGS. 4A and 4B.

Like the embodiment shown in FIGS. 4A and 4B, when the housing 516 is displaced relative to the injection site, the springs 534 deform to allow movement of the housing 516 relative to the injection site whilst allowing the microneedle array 594 to remain in situ.

It will be appreciated that by mounting the support of the embodiments described above on a plurality of springs, the support can be movably mounted relative to the housing to isolate the parenteral interface from impacts to the housing. Although the embodiments described above are depicted with a plurality of helical springs, other springs may also be used. For example, a plurality of leaf springs, conical springs, or other springs may also be used. The use of springs as a resiliently deformable mount between the support and the housing may be particularly well suited to some implementations in which it may desirable for the support to bias the needle assembly towards the skin. For example, in some embodiments, the springs may be configured to bias the support into a position in which the skin facing surface of the support extends beyond the under surface of the housing such that the springs are compressed slightly as the parenteral interface is brought into contact with the injection site. By configuring the device such that the springs are slightly compressed with the device in the position shown in FIG. 5A, the springs can be configured to expand in the event that the housing 516 is pulled away from the injection site, thereby maintaining the depth of insertion of the needle assembly, or at least providing a buffer against withdrawal of the needle assembly from the injection site.

It will also be appreciated that the deformable spring mount described above does not need to be statically mounted respect to the housing. For example, the features described above may be implemented in injection devices comprising a movable needle hub which is configured to advance the needle from a retracted, pre-injection position (in which the needle does not extend from the housing) to an advanced, injection position (in which the needle extends from the housing to penetrate an injection site). In such embodiments, the spring mount may be provided between the support and the needle hub component.

FIGS. 6A and 6B show yet another embodiment of an injection device according to the disclosure. The embodiment shown in FIGS. 6A and 6B show a device 610 which employs an active displacement compensation system for maintaining the parenteral interface in contact with the skin. Like the forgoing figures, FIG. 6A shows the device 610 in a non-displaced position relative to the injection site, while FIG. 6B shows the device 610 as the housing is displaced by an external device.

As shown in FIG. 6A, the device 610 is similar to the devices 210, 310, 410 and 510 described above and comprises a housing 616, an adhesive layer or one or more adhesive patches 622 configured to secure the device 610 against the skin 212 of an injection site.

The device 610 further comprises a cavity 624 which houses the parenteral interface comprising a needle assembly (in this case a hypodermic needle 614) and a support 626, which supports the needle assembly 614. The needle 614 is in fluid communication with a flexible conduit 620, which is configured to deliver medicament from a medicament container to the needle 614.

Instead of a passive parenteral interface positioning system (such as resiliently deformable layer or the springs described above), the device 610 comprises an active parenteral interface positioning system, which comprises one or more actuators 636 coupled to a motor (or motors) and configured to actively control the positioning of the support 626 within the cavity 624. For example, the active parenteral interface positioning system can comprise one or more servo motors configured to drive one or more telescopically extendable actuators 636 configured to advance the support 626 in the z direction relative to the housing 616. The servo motors can be placed in communication with one or more sensors 638 configured to sense detachment of the housing 616 and/or the parenteral interface from the skin 212. A controller (not shown) is configured to control activation of the servo motors and extension of the telescopic actuators in response to sensed detachment of the device 610 from the skin 212.

The sensor(s) 638 can be provided on an underside surface of the housing 616 and configured to sense detachment of the housing 616 from the skin 212. Activation of the actuators 636 to correct for sensed detachment of the housing 616 from the skin 212 is shown schematically in FIG. 6B.

In the embodiment shown in FIGS. 6A and 6B, two actuators 636 are illustrated, each configured to be driven by an associated motor. Two sensors 638 are also provided, one associated with each actuator 636. In this embodiment, sensed detachment of the sensor on the right (see FIG. 6B, detachment indicated with arrow D) results in actuation of the actuator 636 to prevent detachment of the support 626 from the injection site. By actuating one actuator 636 in response to detachment sensed on one side of the housing 616, the parenteral interface, and in particular support 626 can be configured to tilt relative to the housing to compensate for asymmetrical detachment of the housing 616 from the skin surface. Where necessary, suitable mechanical fixation of the support 626 to the actuators 636 can be provided to allow pivoting of the support 626 with respect to the telescopic actuator.

In the embodiment shown in FIGS. 6A and 6B, the sensors 638 are configured as microneedle sensors. Each sensor 638 is configured as a microneedle array fixed to the housing 616 of the device 610. The microneedle array may be configured to characterise the skin-electrode contact force. One method of determining the skin-electrode contact force between a microneedle array sensor 638 and skin (e.g. a skin 212 at an injection site) can be by analysis of signal-to-noise-ratio for ECG signals sensed using the microneedle array as a ‘dry electrode’, which is dependent on contact force between the skin and the microneedle array. One exemplary technique for contact force analysis is described in “Design, fabrication and skin-electrode contact analysis of polymer microneedle-based ECG electrodes”, Journal of Micromechanics and Microengineering, vol. 26 (2016), O'Mahony, Conor, et al., the entire contents of which is hereby incorporated by reference.

The skilled person will understand that other sensors may be used to determine correct placement of the needle assembly with respect to the injection site. For example, in addition (or as an alternative) to microneedle sensors configured as ECG electrodes, capacitive or ‘touch’ sensors may be used to detect contact between the support and the injection site. Displacement sensors may also be used to detect if the support has been displaced from the correct position in which it is contact with the injection site. Microneedle sensors configured to detect contact with the interstitial fluid of the skin can also be used. Other suitable sensors suitable for use in connection with the present invention will be apparent to the person skilled in the art in light of the present disclosure.

It will also be appreciated that although the embodiment shown in FIGS. 6A and 6B comprises two sensors, two motors and two actuators, other combinations are possible. For example, one sensor, one actuator, and one motor may be provided. Alternatively, one, two, three or more sensors, motors and actuators may also be provided. The skilled person will also appreciate that sensors, actuators, and motors need not be provided in a 1:1:1 ratio. Rather, multiple sensors may provide feedback to the controller to cause actuation of one or more actuators. Similarly, multiple actuators may be driven (selectively) by a single motor.

Moreover, although the embodiment shown in FIGS. 6A and 6B comprises a circumferential space 630 between an outer edge of the support 626 and an inner wall 624a of the cavity 624, this arrangement is not necessary in all configurations, as described above.

As an alternative to sensors provided on the housing (or as an addition thereto), sensor(s) 638 can be provided on the support 626 (and optionally as part of a drug delivery microneedle array) such that they are configured to detect detachment of the support 626 from the skin 212, and to advance the support 626 to prevent detachment of the support 626 from the skin 212, thereby maintaining the depth of needle insertion in the injection site.

FIGS. 7A and 7B show another embodiment of an injection device according to the disclosure. The injection device 710 shown in FIGS. 7A and 7B is similar to the injection device 610 shown in FIGS. 6A and 6B. Like FIG. 6A, FIG. 7A shows the device 710 in a rest position (a non-displaced position). FIG. 7B shows the device 710 during an impact that displaces the device 710 relative to the skin 212 of the injection site.

The device 710 comprises a housing 716 configured to be secured in place against the skin using an adhesive layer 722 or adhesive patches. The housing 716 comprises a cavity 724, which houses a parenteral interface comprising a support 726 mounted on at least one actuator 736, driven by a motor in response to sensed detachment of the housing 716 from the skin 212 by sensors 738.

The cavity comprises sidewalls 724a, which are separated from the support 726 by an optional circumferential space 730. Where the device 710 differs from the device 710 of FIGS. 6A and 6B is in the form that needle assembly takes. In the embodiment described with reference to FIGS. 6A and 6B, the parenteral interface comprises a needle 614 mounted on the support 726. However, in the embodiment shown in FIGS. 7A and 7B, the needle assembly takes the form of a microneedle array 794, which comprises a plurality of microneedles configured to deliver medicament to the injection site. The flexible conduit 720 is therefore configured to supply the microneedle array 794 in place of being connected to a single hollow injection needle, as shown in FIGS. 6A and 6B.

Like the embodiment shown in FIGS. 6A and 6B, when the housing 716 is displaced relative to the injection site, the actuators 736 are configured to actively reposition the support 726 relative to the housing in response to sensed detachment of the housing 716 from the skin 212 by sensors 738.

In the embodiments illustrated in FIGS. 6A-7B, the sensors are positioned on the underside of the housing and are configured to sense detachment of the housing relative to the skin to allow the actuators to compensate for sensed displacement of the housing by advancing (or retracting) the parenteral interface relative to the skin to maintain a desired contact pressure and/or insertion depth between the needle assembly and the skin of the injection site. Alternatively or additionally, sensors may be provided on the support of the parenteral interface to directly sense the contact pressure of the parenteral interface against the skin and actively compensate for sensed displacement using the actuators described above.

It will be appreciated that by mounting the support of the embodiments described above on one or more actuators driven by one or motors configured to reposition the parenteral interface relative to the housing in response to detachment of the device from the injection site, the support is movably mounted relative to the housing to isolate the parenteral interface from impacts to the housing. Although the embodiments described above are described with reference to telescopic actuators driven by an associated servo motor, other configurations may also be used. The use of active parenteral interface positioning systems that allow positioning of the parenteral interface relative to the housing may be particularly well suited to some implementations, for example those in which it is desirable for a constant contact pressure to be maintained between the parenteral interface and the injection site. This may be particularly advantageous in the context of wearable devices in which the needle assembly comprises a microneedle array, since the depth of penetration of microneedles is ideally closely controlled throughout the duration of an injection to prevent leakage. By configuring the device such that the contact force between the parenteral interface and the injection site actively maintained, in the event that the housing is pulled away from the injection site, the depth of insertion of the needle assembly can be maintained.

It will also be appreciated that the active parenteral positioning system described above does not need to be statically mounted respect to the housing. For example, the features described above may be implemented in injection devices comprising a movable needle hub which is configured to advance the needle from a retracted, pre-injection position (in which the needle does not extend from the housing) to an advanced, injection position (in which the needle extends from the housing to penetrate an injection site). In such embodiments, the actuators 636 may be provided between the support and the needle hub component. Alternatively, the actuators 636 may be configured to also control insertion of the needle assembly prior to injection.

Turning now to FIGS. 8 and 9, yet further embodiments are described which allow for movement of the parenteral interface relative to the housing.

FIG. 8 shows a device 810 which is similar to the devices 210, 310, 410, 510, 610 and 710 described above. However, instead of using springs, deformable material or actuators to main the position of the parenteral interface relative to the injection site, the embodiment of FIG. 8 comprises a rotationally mounted support for the needle assembly.

As shown in FIG. 8, a device 810 comprises a housing 816 configured to be secured in place against the skin 212 using an adhesive layer 822 or adhesive patches. The housing 816 comprises a cavity 824, which houses a parenteral interface comprising a support 826 rotationally mounted with respect to the housing 816. The pivotal mount can be provided by a gimbal 840 or ball and socket joint. Other rotational joint configurations are possible and can provide a lesser degree of rotational freedom than a ball and socket joint or a gimbal joint. For example, a simple twist joint can be provided to allow rotation of the support 826 relative to the housing 816 about a single axis.

The support 826 is configured for rotation about at least one axis, for example the z axis. Rotationally mounting the support 826 about the z axis relative to the housing 816 ensures that rotation of the housing 816 in the x-y plane relative to the skin does not result in twisting of the needle 814 within the injection site. In at least some embodiments, the support 826 can be configured to rotate about all three axes (x, y, z) to allow the support to pivot within the cavity 824, thereby maintaining contact of the parenteral interface with the skin even if the housing 816 of the device is twisted or lifted from the injection site.

As shown in FIG. 8, the cavity 624 comprises sidewalls 824a, which are separated from the support 826 by an optional circumferential space 830. The circumferential space allows the parenteral interface to tilt and pivot within the cavity 824 to maintain contact with the skin 212 of the injection site.

FIG. 9 shows a device 910, which is similar to device 810 described above. Where the device 910 differs from the device 810 of FIG. 8 is in the form that needle assembly takes. In the embodiment described with reference to FIG. 8, the parenteral interface comprises a needle 814 mounted on the support 826. However, in the embodiment shown in FIG. 9, the needle assembly takes the form of a microneedle array 994, which comprises a plurality of microneedles configured to deliver medicament to the injection site. The flexible conduit 920 is therefore configured to supply the microneedle array 994 in place of being connected to a single hollow injection needle, as shown in FIG. 8.

Like the embodiment shown in FIG. 8, when the housing 916 is displaced relative to the injection site, the support 926 is configured to tilt and/or rotate relative to the housing to maintain contact between the support 926 and the skin 212.

It will be appreciated that by mounting the support of the embodiments rotationally, for example about a pivot point, gimbal or ball and socket mount, as described above, the support is movably mounted relative to the housing to isolate the parenteral interface from impacts to the housing.

It will also be appreciated that the gimbal mount (or other rotational mount for supporting the needle support described above) does not need to be statically mounted respect to the housing. For example, the features described above may be implemented in injection devices comprising a movable needle hub which is configured to advance the needle from a retracted, pre-injection position (in which the needle does not extend from the housing) to an advanced, injection position (in which the needle extends from the housing to penetrate an injection site). In such embodiments, the rotational mount may be provided between the support and the needle hub component.

Turning now to FIGS. 10A and 10B, yet another embodiment is described, which comprises an insertion mechanism. FIGS. 10A and 10B show a device 1010 which is similar to the devices 210, 310, 410, 510, 610, 710, 810 and 910 described above. The device 1010 differs from the devices described above in that it comprises an insertion mechanism configured to advance the needle assembly (here a micro-needle array 1094) from a retracted position (shown in FIG. 10A) in which the needle assembly does not extend from the housing 1016 of the device 1010 to an extended position (shown in FIG. 10B) in which the needle assembly extends from the housing 1016 to make contact with the skin of an injection site.

The insertion mechanism comprises a support base 1052, which is movably mounted with respect to the housing 1016 and is configured to move between a first position (shown in FIG. 10A) to a second position (shown in FIG. 10B) and an actuator 1050 configured to move the support base 1052 between the first and second positions. The actuator 1050 any suitable mechanism for advancing the needle assembly into an injection position. It can comprise a power source such as a mechanical spring or a motor driven actuator configured to move the support base 1052 into the second position.

The support base 1052 is coupled to the parenteral interface 1026 by a plurality of springs 1032 in the configuration shown in FIGS. 10A and 10B. However, the skilled person will recognise that the insertion mechanism described with reference to FIGS. 10A and 10B can be configured to include any of the parenteral interface support arrangements described above with reference to FIGS. 1-9.

The insertion mechanism may be configured to advance the support 1026 into a position in which the parenteral interface extends beyond a lower surface of the housing (as shown in FIG. 10B) when the springs 1032 are uncompressed. This configuration advantageously ensures that the springs 1032 are slightly compressed when the device is secured against the skin of the injection site, thus biasing the parenteral interface towards the skin to maintain contact with the injection site even if the device were to be subject to temporary or extended dislodgement relative to the injection site.

FIGS. 10A and 10B also show, as an optional additional feature, a releasable locking mechanism 1054 configured to maintain the insertion mechanism in an active position (shown in FIG. 10A). The releasable locking mechanism 1054 is here shown as a latch arm configured to engage the support base 1052 to prevent advancement of the support base 1052 to the second position.

As shown in FIG. 10B, the latch arm that forms the releasable locking mechanism 1054 in this configuration is deflectable into an inactive position (see FIG. 10B) in which it allows advancement of the support base 1052 relative to the housing 1016, thereby allowing extension of the parenteral interface and the needle assembly into an injection position. Although a flexible latch arm is shown in FIGS. 10A and 10B, the skilled person will appreciate that other releasable locking arrangements are possible.

Yet another embodiment of the invention will now be described with reference to FIGS. 11A and 11B. FIGS. 11A and 11B show a device 1110 which is similar to the devices 210, 310, 410, 510, 610, 710, 810 and 910 described above. The device 1110 differs from the devices described above in that it comprises a deployment mechanism 1160 configured to deploy the needle assembly (here a micro-needle array 1194) into an injection position. As shown in FIGS. 11A and 11B, the deployment mechanism is configured to move the needle assembly from a retracted position in which does not extend from the housing 1116 of the device 1110 (see FIG. 11A) to an extended position in which the needle assembly extends from the housing 1016 to make contact with the skin of an injection site (see FIG. 11B).

The deployment mechanism 1160 here takes the form of a manual deployment actuator configured to allow the user to advance the parenteral interface comprising the needle assembly (here microneedle array 1194) into an injection position. The deployment mechanism 1060 takes the form of a key that engages, at a first end 1162, the support 1126 for the parenteral interface. The key extends through a channel in the housing 1116 to a second end, which comprises an actuation member 1164 or handle. The deployment mechanism 1160 is slidable within the housing between a first position (shown in FIG. 11A) to a second position (shown in FIG. 11B). Because the first end 1162 of the key is engaged with the support 1126, movement of the actuation member 1164 to the position shown in FIG. 11B brings the needle assembly (here microneedle array 1194) into contact with the injection site.

In the configuration shown in FIGS. 11A and 11B, the support 1126 is mounted within the housing on a support base 1152 by a plurality of springs 1132. However, the skilled person will appreciate that the deployment mechanism 1060 described with reference to FIGS. 11A to 11B can be implemented in any of the embodiments described above.

Moreover, it will be appreciated that the deployment mechanism 1160 can be configured to maintain the support 1126 in the retracted position shown in FIG. 11A, with the springs 1132 compressed between the support 1126 and the support base 1152. In the position shown in FIG. 11B, the springs 1132 are still compressed, although to a lesser degree than in the position shown in FIG. 11A, thereby maintaining the parenteral interface in contact with the skin of the injection site.

To avoid accidental retraction of the parenteral interface from the injection site after deployment of the needle assembly into the injection position, the deployment mechanism 1160 can be configured to be removable from the device 110. For example, the user may be able to slide the first end 1162 of the key out of engagement with the support 1126 and withdraw the key from the housing 1116 once the parenteral interface has been deployed.

Although the deployment mechanism described with reference to FIGS. 11A and 11B takes the form of a manually actuated key configured to move the support 1126 from a first position to a second position, the skilled person will appreciated that other configurations are also possible. For example, instead of a manually actuated deployment mechanism, an automated deployment mechanism may comprise a power source (mechanical or electrically driven) to advance the support 1126 into the injection position shown in FIG. 11B. In the embodiments described above, each of the devices 210, 310, 410, 510, 610, 710, 810, 910, 1010, and 1110 comprise a configuration which allows movement of the parenteral interface relative to the housing in response to movement of the device relative to the injection site. However, it will be appreciated that the movable parenteral mounts described above can be combined in some embodiments. For example, a passively compensating parenteral mount (e.g. a support mount comprising a deformable material, a plurality of springs, or a pivot mount) may be combined with the active compensation mechanisms described above. For example, a rotationally mounted support, configured to rotate about at least the z axis, may be combined with the active, motor driven parenteral interface supports described above. Since a telescopic actuator cannot compensate for rotational movement of the housing about the z axis (e.g. a as result of twisting a device as shown in FIG. 10), active compensation mechanisms, such as the ones shown in FIGS. 6A-7B, may be combined with a rotationally mounted support of the type shown in FIG. 8 or 9.

In each of the embodiments described above, an adhesive layer or adhesive patches have been described as provided on an underside of the housing for securing the device to the skin. However, it will be appreciated that wearable devices may be affixed to the skin in different ways. For example, separate adhesive may be used to secure the device in place. Adhesive may also be provided on the parenteral interface to maintain the interface in position relative to the injection site. This can be provided as an addition to securing features provided on the housing of a wearable device.

In any of the embodiment described above comprising sensors configured to detect displacement of the device, the controller can be further configured to gather data regarding the placement and positioning of microneedles, or the delivery pattern of medicament through the device.

For simplicity, the embodiments described above have been illustrated in the context of a wearable drug delivery device configured as a self-contained unit comprising a medicament container, and a drive system for delivering medicament from a container through a conduit to the needle assembly. However, it will be appreciated that the advantages associated with the embodiments described above also apply to wearable devices configured for use with external medicament containers, and also to handheld autoinjectors, an in particular those that are designed to be held against the skin to deliver a dose of medicament for an extended injection period, for example 10-120 seconds.

In addition to the devices described above, the present disclosure also provides a number of exemplary methods. In particular, the present disclosure provides methods of manufacturing devices according to any of the embodiments described above, and exemplary methods for supporting a needle of an injection device. The methods for supporting a needle assembly of an injection device include methods for supporting the needle assembly in an injection position in preparation for injection, and for supporting a needle assembly of an injection device in an injection position between delivery of doses of medicament. The latter is particularly applicable to wearable devices configured to deliver discrete bolus doses of medicament, spaced out over extended periods of time.

In one embodiment, a method of supporting a needle assembly of an injection device in preparation for injection of a medicament includes: placing, against an injection site, an injection device comprising a housing having a cavity, a parenteral interface arranged within the cavity, wherein the parenteral interface comprises a support and a needle assembly mounted to the support and configured to deliver a dose of medicament to an injection site, wherein the support is mounted on a plurality of springs extending from a spring base within the housing, the plurality of springs having non-coincidental longitudinal axes. The method further includes fixing the injection device to the injection site using an adhesive provided on the housing; and compressing the springs between the support and the spring base.

The method described above may further comprise the step of moving the parenteral interface relative to the housing by compressing and/or extending at least one of the plurality of springs to maintain contact between the parenteral interface and the skin.

In another embodiment, a method of supporting a needle assembly of an injection device in preparation for injection of a medicament comprises placing, against an injection site, a device comprising a housing having a cavity, a parenteral interface arranged within the cavity, wherein the parenteral interface comprises a support and a needle assembly mounted to the support and configured to deliver a dose of medicament to an injection site, wherein the support is mounted within the cavity on a resiliently deformable mount coupled to the housing and wherein: the support comprises an outer sidewall; the cavity comprises an inner sidewall, and the outer sidewall of the support is separated from the inner sidewall of the cavity by a circumferential space extending around the outer sidewall of the support. The method further comprises fixing the injection device to the injection site using an adhesive provided on the housing and compressing the resiliently deformable material between the support and the housing.

The method described above may further comprise the step of moving the parenteral interface relative to the housing by compressing and/or expanding the resiliently deformable mount to maintain contact between the parenteral interface and the skin.

In yet another embodiment, a method of supporting a needle assembly of an injection device in preparation for injection of a medicament includes: placing, against an injection site, an injection device comprising: a housing having a cavity, a parenteral interface arranged within the cavity, wherein the parenteral interface comprises a support and a needle assembly mounted to the support and configured to deliver a dose of medicament to an injection site, a flexible conduit configured to deliver medicament from the medicament container to the needle assembly, wherein the support is movably mounted within the cavity on a plurality of motors configured to maintain contact between the parenteral interface with an injection site. The method further includes the steps of fixing the injection device to the injection site using an adhesive provided on the housing, and maintaining contact between the parenteral interface and the injection site by actuation of at least one motor.

The method described above may further comprise sensing a contact force between the housing and the skin and/or between the parenteral interface and the skin and actuating the actuators in response to sensed displacement of the parenteral interface from a predetermined needle penetration depth.

In yet another embodiment, a method of supporting a needle assembly of an injection device in preparation for injection of a medicament includes: placing, against an injection site, a device comprising: a housing having a cavity, a parenteral interface arranged within the cavity, wherein the parenteral interface comprises a support and a needle assembly mounted to the support and configured to deliver a dose of medicament to an injection site, wherein a flexible conduit is provided in fluid communication with the medicament container and the needle assembly, and wherein the support is rotationally mounted with respect the housing to allow rotation of the support within the cavity. The method further comprises fixing the injection device to the injection site using an adhesive provided on the housing, and bringing the parenteral interface into contact with an injection site.

The method described above may further comprise the step of moving the parenteral interface relative to the housing by rotating the support about a pivot mount to maintain contact between the parenteral interface and the skin.

The preceding detailed description describes systems and methods for supporting a parenteral interface in an injection position. However, the skilled person will understand that the invention is not limited to use in connection with the exemplary device described here. Rather, one or more benefits associated with the present invention may be implemented in connection with other drug delivery systems, as will be apparent to the skilled person in light of the preceding detailed description.

It will also be understood that, where used, the terms “proximal”, “distal”, “front”, “back”, “side”, “top” and “bottom” are used for convenience in interpreting the drawings and are not to be construed as limiting. The term “comprising” should be interpreted as meaning “including but not limited to”, such that it does not exclude the presence of features not listed.

The embodiments described and shown in the accompanying drawings above are provided as examples of ways in which the invention may be put into effect and are not intended to be limiting on the scope of the invention. Modifications may be made, and elements may be replaced with functionally and structurally equivalent parts, and features of different embodiments may be combined without departing from the disclosure.

1. An injection device comprising:
a housing configured to receive a medicament container;
a parenteral interface comprising a support and a needle assembly mounted to the support and configured to deliver a dose of medicament to an injection site, the support being arranged in a cavity within the housing; and
a flexible conduit configured to deliver medicament from the medicament container to the needle assembly,
wherein the support is mounted on a plurality of springs coupled to a spring base provided in the housing, each spring having a longitudinal axis, and
wherein the longitudinal axes are non-coincident.

2. The injection device according to claim 1, wherein the needle assembly comprises a hollow injection needle or a microneedle array.

3. (canceled)

4. The injection device according to claim 1, wherein the plurality of springs comprises at least one helical spring.

5. The injection device according to claim 1, wherein:
the support comprises an outer sidewall,
the cavity comprises an inner sidewall, and
the outer sidewall of the support is separated from the inner sidewall of the cavity by a circumferential space extending around the outer sidewall of the support.

6. The injection device according to claim 1, wherein the spring base is fixedly mounted within the housing, movably mounted within the housing, rotatably mounted in the housing, or pivotably mounted the housing.

7-9. (canceled)

10. The injection device of claim 6, wherein the spring base is configured to be movably mounted within the housing for travel in a distal direction to advance the needle assembly from a retracted position to an extend position from the housing.

11. The injection device according to claim 1, wherein the plurality of springs are configured to bias the support into a position in which the needle assembly extends beyond a skin contacting surface of the housing.

12. The injection device according to claim 1, wherein the injection device further comprises an insertion mechanism configured to move the spring base between:
a first position relative to the housing in which the needle assembly does not extend from the housing, and
a second position relative to the housing, in which the needle assembly extends from the housing for insertion into the injection site.

13. The injection device according to claim 1, further comprising a releasable locking mechanism configured to:
maintain the support or the spring base in a first position relative to housing, in which the needle assembly does not extend from the housing, when the locking mechanism is in an active state; and
allow movement of the support or the spring base to a second position, in which the needle assembly extends from the housing, when the locking mechanism is in an inactive state.

14. The injection device according to claim 13 wherein the releasable locking mechanism configured to:
maintain the support in the first position relative to the spring base in which the plurality of springs are compressed, and in which the needle assembly does not extend from the housing, when the locking mechanism is in the active state; and
allow movement of the support relative to the spring base under influence of the plurality of springs to the second position, in which the needle assembly extends from the housing, when the locking mechanism is in the inactive state.

15. The injection device according to claim 1, further comprising a deployment mechanism coupled to the support, wherein the deployment mechanism is configured to move the support between a first position in which the needle assembly does not extend from the housing and a second position in which the needle assembly extends from the housing.

16. (canceled)

17. The injection device according to claim 1, wherein the housing includes a skin contacting surface, the skin contacting surface comprising an adhesive portion configured to attach the injection device to the skin of a user.

18-20. (canceled)

21. An injection device comprising:
a housing configured to receive a medicament container;
a parenteral interface comprising a support and a needle assembly mounted to the support and configured to deliver a dose of medicament to an injection site, the support being arranged in a cavity within the housing; and
a flexible conduit configured to deliver medicament from the medicament container to the needle assembly,
wherein the support is movably mounted within the cavity, and
wherein a plurality of motors is configured to move the support with respect to the housing to maintain the parenteral interface in contact with the injection site.

22. The injection device according to claim 21, wherein the needle assembly comprises a hollow injection needle or a microneedle array.

23. (canceled)

24. The injection device according to claim 21, wherein each motor of the plurality of motors is a servo motor.

25. (canceled)

26. The injection device according to claim 21, wherein each of the plurality of motors is in communication with at least one sensor configured to sense detachment of the parenteral interface from the injection site.

27. The injection device according to claim 26, wherein the at least one sensor is a microneedle electrode sensor.

28. The injection device according to claim 26, wherein the at least one sensor is provided on a skin contacting surface or on a skin facing surface of the support.

29. (canceled)

30. The injection device according to claim 21, wherein:
the support comprises an outer sidewall,
the cavity comprises an inner sidewall, and
the outer sidewall of the support is separated from the inner sidewall of the cavity by a circumferential space extending around the outer sidewall of the support.

31. The injection device according to claim 21, wherein the motors are mounted to a motor base movably mounted within the housing, rotatably mounted in the housing, or pivotably mounted in the housing.

32-33. (canceled)

34. The injection device of claim 31, wherein the motor base is movably mounted within the housing for travel in a distal direction to advance the needle assembly from a retracted position to an extended position.

35. The injection device according to claim 21, wherein the plurality of motors are configured to maintain the support in a position in which the needle assembly extends beyond a skin contacting surface of the housing.

36. The injection device according to claim 35, wherein the skin contacting surface further comprises an adhesive portion configured to attach the injection device to the skin of a user.

37-80. (canceled)
